(19) United States
(12) Patent Application Publication
(10) Pub. No.: US 2006/0188400 A1
(43) Pub. Date: Aug. 24, 2006

(54) Thermal-type infrared detection element

(75) Inventor: Tokuhito Sasaki, Tokyo (JP)

Correspondence Address: MCGINN INTELLECTUAL PROPERTY LAW GROUP, PLLC, 8321 OLD COURTHOUSE ROAD, SUITE 200, VIENNA, VA, 22182-3817, US

(73) Assignee: NEC Corporation, Tokyo (JP)

(21) Appl. No.: 11/356,379

(22) Filed: Feb. 17, 2006

(30) Foreign Application Priority Data
Feb. 18, 2005 (JP) 2005-042222

(51) Int. Cl. G01N 33/00

(52) U.S. Cl. 422/82.02

(57) ABSTRACT

A convex pattern in which a plurality of substantially congruent projections composed of an infrared absorbing material are arranged at a substantially constant pitch is formed on the side of a photoreceptor that is irradiated with infrared rays, or a concave pattern in which a plurality of substantially congruent holes are arranged at a substantially constant pitch is formed in an infrared absorption film disposed on the side irradiated with infrared rays, and the infrared rays incident on the photoreceptor are dispersed by the convex pattern or concave pattern. By this configuration, reflection of infrared rays is minimized, the absorption efficiency of infrared rays is increased, and the sensitivity of the thermal-type infrared detection element is enhanced. This convex pattern can be formed using a common semiconductor manufacturing device, and has excellent adhesion to the immediately underlying infrared absorption film. The reliability and uniformity of the thermal-type infrared detection element can therefore be enhanced.

### BACKGROUND OF THE INVENTION

1. Field of the Invention

The present invention relates to a thermal-type infrared detection element, and particularly relates to a structure for an infrared absorption body that constitutes the photoreceptor of a thermal-type infrared detection element.

2. Description of the Related Art

A thermal-type infrared detection element measures the temperature of an object from the change in resistance that occurs in a heat-sensitive resistor. This usually occurs when infrared rays emitted by a body are absorbed and converted to heat in an infrared absorption body, the temperature of a bolometer thin film or other heat-sensitive resistor that forms a diaphragm having a microbridge structure is increased, and the resistance thereof is changed.

More specifically, this type of thermal-type infrared detection element is composed of a photoreceptor 11 provided with a bolometer layer 6 and an infrared absorption body (infrared absorption films 5, 7, and 9) for absorbing incident infrared rays and protecting the bolometer layer 6, and is also composed of a beam 10 provided with wiring 8 for connecting the bolometer layer 6 with a reading circuit 2 formed in advance on the circuit board 1. This beam 10 holds the photoreceptor 11 in a suspended state above the circuit board 1. When incident infrared rays are absorbed by the infrared absorption body, and the temperature of the photoreceptor 11 is increased, the resistance of the bolometer layer 6 changes, and this change in resistance is detected by the reading circuit 2 and outputted as a temperature. Thermal-type infrared detection elements having this type of structure are disclosed in JP-A 2002-71452 (pp. 5-8, FIG. 6) and other publications, for example.

Increasing the change in resistance of the bolometer layer 6 with respect to the change in temperature of the photoreceptor 11 is of primary importance in increasing the sensitivity (S/N ratio) of the thermal-type infrared detection element described above. Therefore, a material having a large temperature coefficient of resistance (TCR: Temperature Coefficient Resistance) is used as the bolometer layer 6. Increasing the efficiency with which incident infrared rays are absorbed is the second most important factor. In order to achieve this object, an infrared reflection film 3 is provided in a position facing the photoreceptor 11 on the circuit board 1, and the gap between the photoreceptor 11 and the infrared reflection film 3 is set so that an optical resonance structure is formed.

Thermal-type infrared detection elements in which the infrared absorption body that constitutes the photoreceptor 11 has a characteristic structure have been proposed in order to further enhance sensitivity. For example, a thermal-type infrared detection element has also been proposed in which eaves 17 whose central portion is connected to the photoreceptor 11 and whose edges extend so as to cover the beam 10 are provided to the surface of the photoreceptor 11 on which infrared rays are incident, as shown in FIG. 2. In this structure, since infrared rays incident on the beam 10 outside the photoreceptor 11 can be absorbed by the eaves 17, the sensitivity can be enhanced. However, since the heat capacity of the photoreceptor 11 is significantly increased by forming the eaves 17, the temperature change with respect to the incident infrared rays becomes small. The edges of the eaves 17 are also suspended in the air, and are structurally susceptible to impact, vibration, or other disturbances. This structure is therefore not well-suited for a thermal-type infrared detection element used in a harsh environment.

A thermal-type infrared detection element is shown in FIG. 3 as another example of the structure of the infrared absorption body, in which fine particles 18 of gold black, carbon black, or the like are bonded to the outermost layer (on the third infrared absorption film 9 herein) of the photoreceptor 11. In this structure, reflection of infrared rays on the flat surface of the third infrared absorption film 9 is minimized, and the absorption rate of infrared rays can be increased, but such fine particles 18 have poor adhesion, and are easily peeled off in subsequent processing. Significant process limitations therefore occur, and as with the abovementioned structure, this structure is not well-suited for a thermal-type infrared detection element used in a harsh environment, due to the susceptibility of the structure to impact, vibration, and other disturbances. Since the distribution of the fine particles 18 is also not uniform between and within elements, drawbacks occur whereby fluctuations easily occur in the infrared absorption characteristics. Furthermore, a specialized or dedicated manufacturing apparatus is needed for bonding the fine particles 18, which creates the drawback of increasing the cost of the thermal-type infrared detection element.

### SUMMARY OF THE INVENTION

An object of the present invention is to provide a structure for a thermal-type infrared detection element; specifically, a structure for the infrared absorption body that constitutes the photoreceptor, whereby the absorption efficiency of incident infrared rays is increased, and sensitivity is enhanced.

A thermal-type infrared detection element according to the present invention has a substrate, a photoreceptor having a heat-sensitive resistor and an infrared absorption body, and a beam for holding the photoreceptor in midair, in which one end of the beam is fixed to the substrate, wherein a convex pattern in which a plurality of substantially congruent projections composed of an infrared absorbing material are arranged at a substantially constant pitch is formed on the side of the photoreceptor.

Another thermal infrared detection element according to the present invention has a substrate; a photoreceptor having a heat-sensitive resistor and an infrared absorption body; and a beam having wiring whose one end is connected to the heat-sensitive resistor and whose other end is connected to a circuit formed in a substrate, for holding the photoreceptor in midair; wherein the infrared absorption body comprises a first infrared absorption film formed in the bottom layer of the heat-sensitive resistor; a second infrared absorption film formed in the top layer of the heat-sensitive resistor; a third infrared absorption film formed in the top layer of the wiring connected to the heat-sensitive resistor via a through-hole provided to the second infrared absorption film; and a convex pattern formed in the top layer of the third infrared absorption film, wherein a plurality of substantially congruent projections composed of an infrared absorbing material are arranged at a substantially constant pitch.

It is preferred in the present invention that the height-to-width ratio of the projections be 1 or higher, and that the interval between adjacent projections be smaller than the aforementioned height.

Yet another thermal-type infrared detection element of the present invention has a substrate, a photoreceptor having a heat-sensitive resistor and an infrared absorption body, and a beam for holding the photoreceptor in midair, in which one end [of the beam] is fixed to the substrate, wherein a concave pattern in which a plurality of substantially congruent holes are arranged at a substantially constant pitch is formed in an infrared absorption film disposed on the side of the photoreceptor that is irradiated with infrared rays.

Still another thermal-type infrared detection element of the present invention has a substrate, a photoreceptor having a heat-sensitive resistor and an infrared absorption body, and a beam having wiring whose one end is connected to the heat-sensitive resistor and whose other end is connected to a circuit formed in a substrate so that the photoreceptor is held in midair by the beam, wherein the infrared absorption body comprises a first infrared absorption film formed in the bottom layer of the heat-sensitive resistor, a second infrared absorption film formed in the top layer of the heat-sensitive resistor, and a third infrared absorption film that is formed in the top layer of the wiring connected to the heat-sensitive resistor via a through-hole provided to the second infrared absorption film, and that is provided with a concave pattern in which a plurality of substantially congruent holes are arranged at a substantially constant pitch.

It is preferred in the present invention that the depth-to-width ratio of the holes be 1 or higher, and that the interval between adjacent holes be smaller than the aforementioned depth.

Thus, in the present invention, since a convex or concave pattern in which a plurality of substantially congruent projections or holes are arranged at a substantially constant pitch is formed on the surface of the infrared-incident side of the photoreceptor of the thermal-type infrared detection element, reflection of incident infrared rays on the photoreceptor is minimized, and the absorption efficiency of infrared rays can be increased. The sensitivity of the thermal-type infrared detection element can thereby be enhanced.

As described above, the absorption efficiency of infrared rays incident on the photoreceptor can be increased by the thermal-type infrared detection element of the present invention, and the sensitivity of the thermal-type infrared detection element can thereby be enhanced.

The reason for this is that a convex pattern in which a plurality of substantially congruent projections composed of an infrared absorbing material are arranged at a substantially constant pitch is formed on the surface of the infrared-incident side of the photoreceptor of the thermal-type infrared detection element, infrared rays incident on the photoreceptor are scattered by this convex pattern, and reflection can be minimized. Another reason for these effects is that a concave pattern in which a plurality of substantially congruent holes are arranged at a substantially constant pitch is formed in an infrared absorption film disposed on the side of the photoreceptor that is irradiated with infrared rays, infrared rays incident on the photoreceptor are scattered by this concave pattern, and reflection can be minimized.

### BRIEF DESCRIPTION OF THE DRAWINGS

FIG. 1 is a sectional view showing the structure of a conventional thermal-type infrared detection element;

FIG. 2 is a sectional view showing the structure of a conventional thermal-type infrared detection element in which eaves are formed in the photoreceptor;

FIG. 3 is a sectional view showing the structure of a conventional thermal-type infrared detection element in which fine particles are bonded to the photoreceptor;

FIG. 4 is a sectional view in which the structure of one pixel of the thermal-type infrared detection element according to a first embodiment of the present invention is schematically depicted in alignment with the current path;

FIG. 5 is a schematic plane view showing the structure of the convex pattern according to the first embodiment;

FIG. 6 is a schematic perspective view showing the structure of the convex pattern according to the first embodiment;

FIG. 7 is a sectional view showing variation in the structure of the convex pattern according to the first embodiment;

FIGS. 8 through 15 are sectional views showing the sequence of steps in the method for manufacturing the thermal-type infrared detection element according to the first embodiment;

FIG. 16 is a sectional view showing another structure of the thermal-type infrared detection element according to the first embodiment of the present invention;

FIG. 17 is a sectional view in which the structure of one pixel of the thermal-type infrared detection element according to a second embodiment of the present invention is schematically depicted in alignment with the current path;

FIG. 18 is a schematic plane view showing the structure of the concave pattern according to a second embodiment of the present invention;

FIG. 19 is a schematic perspective view showing the structure of the concave pattern according to the second embodiment;

FIG. 20 is a sectional view showing variation in the structure of the concave pattern according to the second embodiment; and

FIG. 21 is a sectional view showing another structure of the thermal-type infrared detection element according to the second embodiment.

### DESCRIPTION OF THE PREFERRED EMBODIMENTS

As described in relation to the prior art, measures must be taken to increase the absorption efficiency of incident infrared rays in order to enhance the sensitivity of the thermal-type infrared detection element. However, drawbacks occur in a structure in which eaves are provided to the surface of the infrared-incident side of the photoreceptor in that the heat capacity of the photoreceptor is increased, and the reliability thereof declines due to susceptibility to vibration or impact. In a structure in which fine particles are bonded to the side of the photoreceptor irradiated with infrared rays, the adhesion strength of the fine particles is low. Therefore, subsequent processing is limited and other significant process limitations are imposed, and specialized equipment is needed. This structure also suffers from drawbacks of low reliability due to the susceptibility to vibration or impact, and the infrared absorption characteristics are prone to fluctuate.

Therefore, the present invention provides a method whereby a thermal-type infrared detection element can easily be formed using a general-use semiconductor manufacturing apparatus capable of micromachining, in which there are no process limitations, and in which high reliability and uniformity can be achieved. In this method, a convex pattern in which a plurality of substantially congruent projections composed of an infrared absorbing material are arranged at a substantially constant pitch is formed on the side of the photoreceptor that is irradiated with infrared rays of the thermal-type infrared detection element, or a concave pattern in which a plurality of substantially congruent holes are arranged at a substantially constant pitch is formed on an infrared absorption film disposed on the side irradiated with infrared rays. Infrared rays incident on the photoreceptor are thereby efficiently absorbed and prevented from reflecting, and the sensitivity of the thermal-type infrared detection element is enhanced. A thermal-type infrared detection element provided with this type of infrared absorption body will be described hereinafter with reference to the drawings.

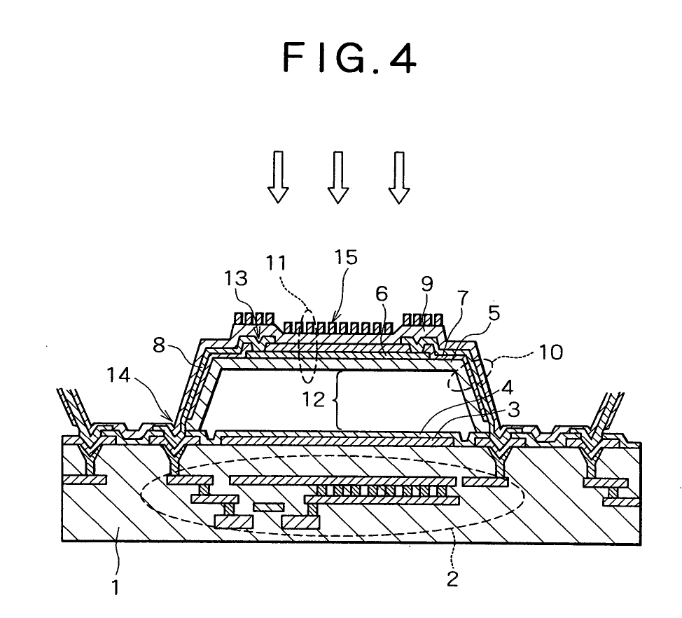

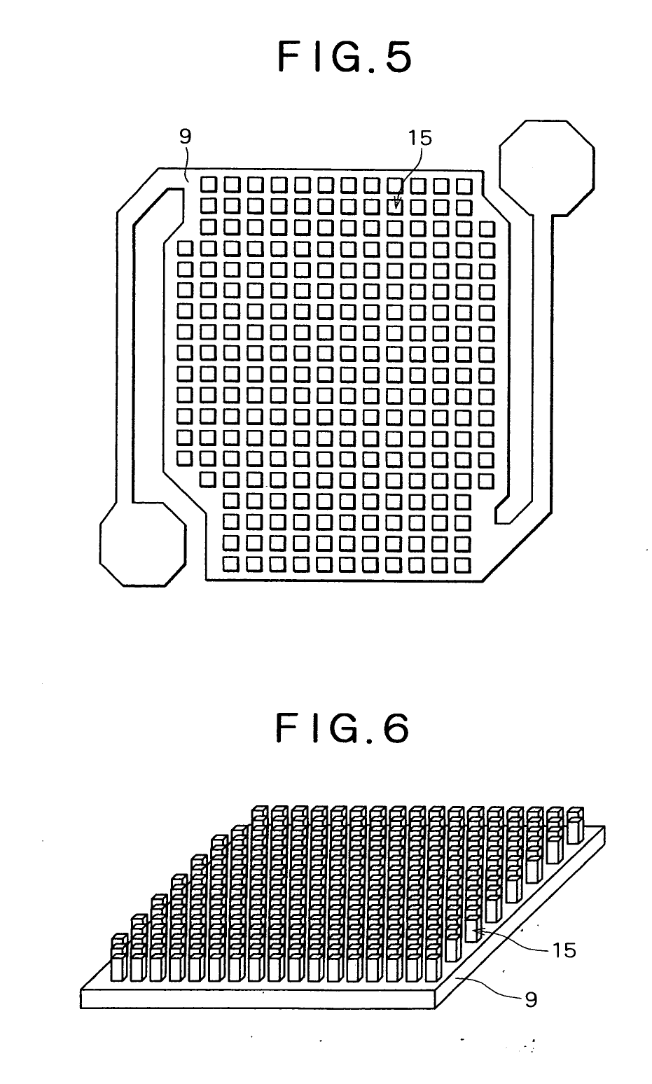

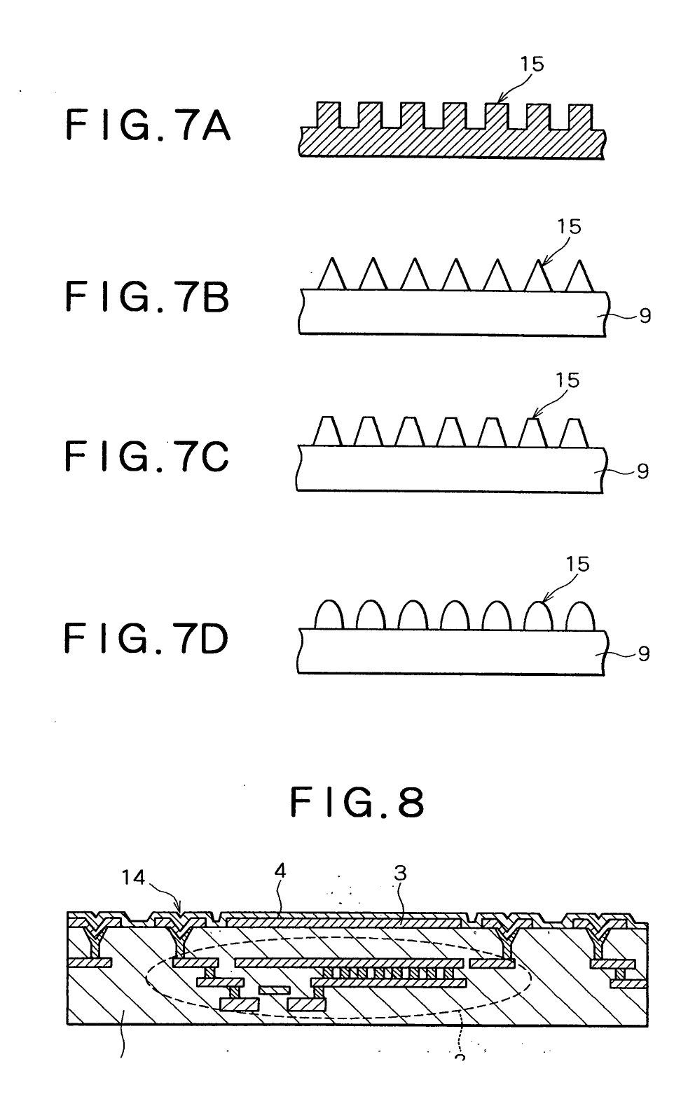

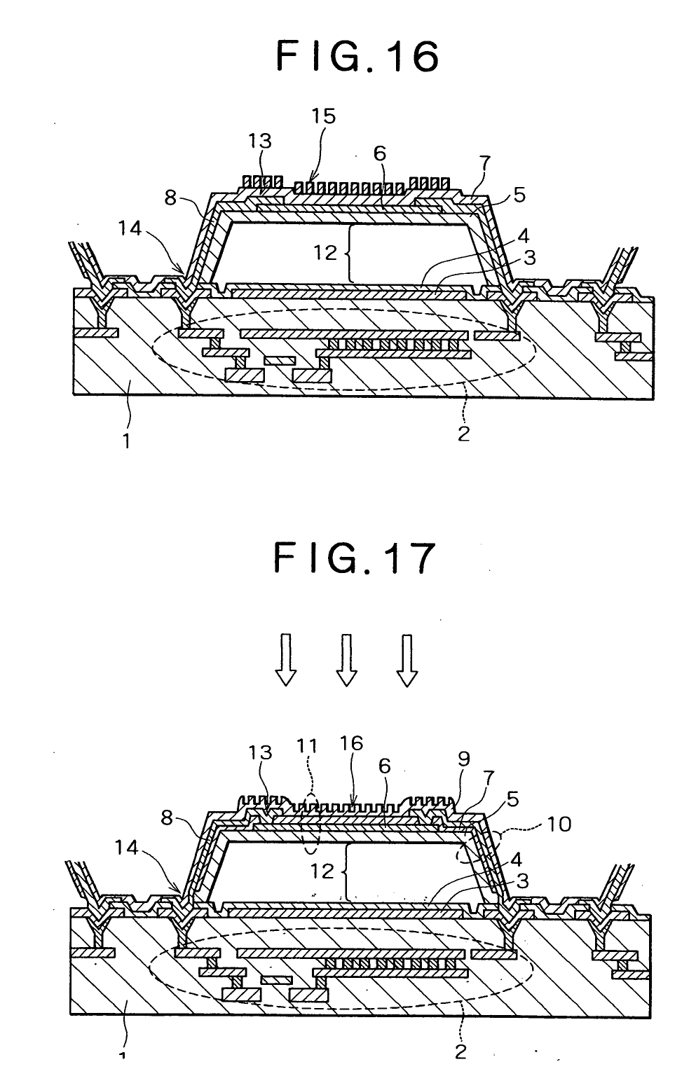

The thermal-type infrared detection element according to a first embodiment of the present invention will first be described with reference to FIGS. 4 through 16. FIG. 4 is a sectional view in which one pixel of the thermal-type infrared detection element of the present embodiment is depicted in alignment with the current path; FIG. 5 is a schematic plane view showing the structure of the convex pattern; and FIG. 6 is a perspective view of the same. FIG. 7 is a sectional view showing variation in the convex pattern; and FIGS. 8 through 15 are sectional views showing the sequence of steps in the method for manufacturing the thermal-type infrared detection element according to the present embodiment. FIG. 16 is a sectional view showing another structure of the thermal-type infrared detection element according to the present embodiment.

In the thermal-type infrared detection element of the present embodiment as shown in FIG. 4, an infrared reflection film 3 composed of Al, Ti, W, or a silicide of any of these elements in the form of a film or the like is formed on a circuit board 1 in which a reading circuit 2 is incorporated into a silicon wafer or other semiconductor wafer by a CMOS process; and a protective film 4 composed of a silicon oxide film, a silicon nitride film, a silicon oxynitride film, or the like is formed on the top layer thereof. A hollow portion 12 is filled with a patterned photosensitive polyimide in an intermediate stage of the device manufacturing, and is removed by oxygen plasma ashing or the like in the final step of device manufacturing. The layer formed by filling in this hollow portion 12 is generally referred to as the sacrifice layer. A photoreceptor 11 is formed on this sacrifice layer, and a beam 10 connected to the end portion of the photoreceptor 11 is formed on the side surface of the sacrifice layer.

The photoreceptor 11 is composed, for example, of a bolometer layer 6 composed of vanadium oxide or the like, and an infrared absorption body (in this case, a first infrared absorption film 5, a second infrared absorption film 7, a third infrared absorption film 9, and a convex pattern 15) formed from a material (SiO, SiN, SiC, SiON, SiCN, SiCO, and the like) for absorbing infrared rays in the waveband from 8 to 14 μm. The beam 10 is composed of wiring 8 composed of Ti or the like, and a protective film (in this case, the first infrared absorption film 5, the second infrared absorption film 7, and the third infrared absorption film 9) for protecting this wiring 8, and holds the photoreceptor 11 in midair over the circuit board 1 via the hollow portion 12, thereby creating a thermal separation structure.

It is sufficient as long as the bolometer material is a material having a high temperature coefficient of resistance (TCR), and any of the following may be used besides vanadium oxide: a NiMoCo oxide, a Ti metal thin film, a polycrystalline silicon thin film, a non-crystalline silicon thin film, a non-crystalline germanium thin film, a non-crystalline silicon germanium thin film, a (La, Sr)MnO₃ thin film, a YBaCuO thin film, or the like. It is also sufficient as long as the wiring 8 has a low coefficient of thermal conductivity, and besides Ti, a Ti alloy or NiCr may be used. A component in which boron or arsenic is implanted/diffused at high concentration in silicon may also be used instead of the wiring 8 when polycrystalline silicon or non-crystalline silicon is used in the bolometer material.

Infrared rays that are incident through the atmospheric window waveband of 8 to 14 μm are absorbed by the infrared absorption body and cause the temperature of the photoreceptor 11 to increase. The resistance of the bolometer layer 6 changes in conjunction with the temperature increase of the photoreceptor 11, and this change in resistance of the bolometer layer 6 is detected by the reading circuit 2 connected via an electrode unit 13, the wiring 8, and a contact unit 14.

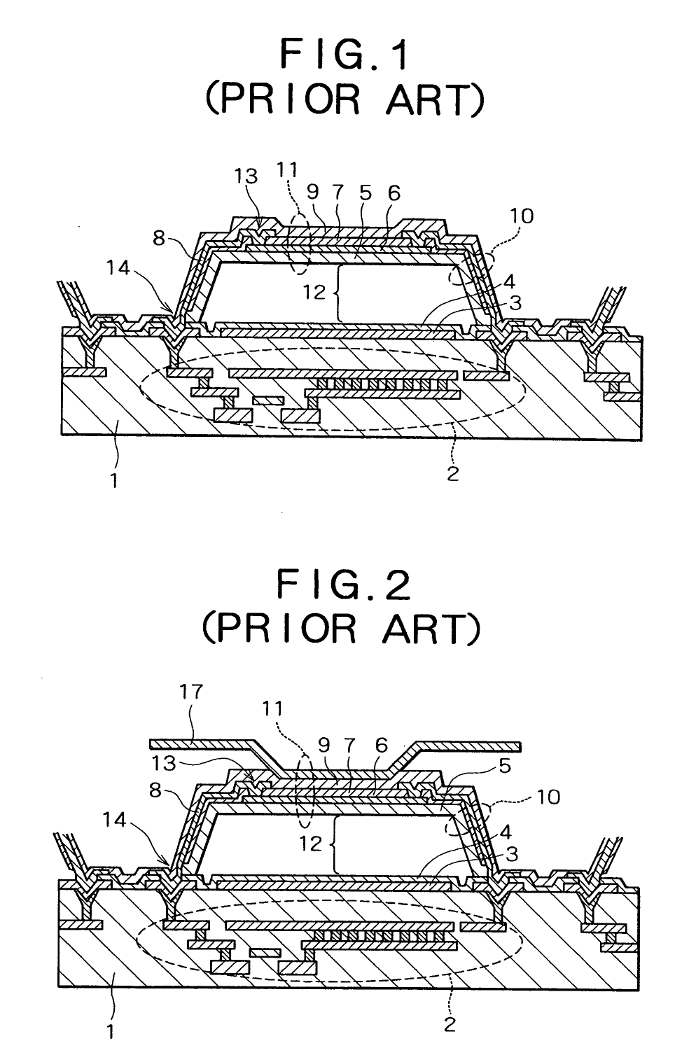

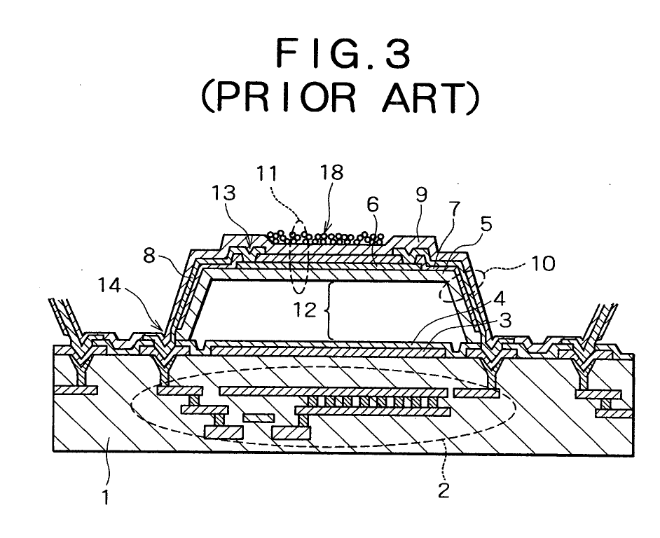

In the conventional thermal-type infrared detection element, the infrared absorption body is formed only by a flat film as shown in FIG. 1. Accordingly, incident infrared rays are easily reflected by the infrared absorption film on the outermost layer, and drawbacks occur whereby infrared rays incident on the photoreceptor 11 cannot be efficiently absorbed. Therefore, in the present embodiment, the convex pattern 15 composed of an infrared absorbing material is formed on the side of the photoreceptor 11 that is irradiated with infrared rays, and the incident infrared rays are dispersed and prevented from reflecting using this convex pattern 15. The infrared absorption efficiency is thereby increased.

A schematic representation of this convex pattern 15 is shown in FIGS. 5 and 6, and this pattern is formed by a plurality of substantially congruent (rectangular column-shaped in this case) projections arranged in a matrix at a substantially constant pitch. This convex pattern 15 can be formed by various methods, and can easily be formed, for example, by depositing an infrared absorbing material onto the infrared absorption film (third infrared absorption film 9) of the outermost layer, forming a resist pattern (a rectangular pattern in this case) thereon in a prescribed shape, and dry-etching the infrared absorbing material using the resist pattern as a mask.

The shape or pitch of the projections may be set as appropriate, but it is preferred in order to increase the infrared absorption efficiency that the aspect ratio (height/width ratio of the projections) be increased and that the interval between adjacent projections be reduced within ranges that allow formation by common photolithography techniques and dry etching techniques. It has been confirmed according to the knowledge of the present applicant that the infrared absorption efficiency is dramatically improved by making the aspect ratio equal to 1 or higher, and making the interval between adjacent projections smaller than the height of the projections. Citing specific numerical values, since a 0.5-μm line-and-space can be formed when an i-line stepper is used, a structure having good infrared absorption characteristics can be obtained by arranging 20×20 to 50×50 projections having a height of approximately 0.5 μm or greater and a width of approximately 0.5 μm at a pitch of approximately 0.5 μm when the edge length of one pixel is approximately 20 to 50 μm.

A convex pattern 15 composed of isolated projections is formed by dry-etching the infrared absorbing material deposited on the infrared absorption film (the third infrared absorption film 9 in this case) of the outermost layer in FIGS. 4 through 6, but this convex pattern 15 may also be formed in the top-layer infrared absorption film itself. For example, as shown in FIG. 7A, the convex pattern 15 may be formed in the top-layer infrared absorption film itself by forming a prescribed resist pattern and etching the exposed portions partway after the top-layer infrared absorption film is formed. Since the surface area of contact with the structural component (the second infrared absorption film 7 in this case) directly below is increased, and adhesion thereto is enhanced in this structure, the ability to withstand vibration or impact can be further increased.

Each projection that constitutes the convex pattern 15 is a square column in FIGS. 5 and 6, but the shape of the projections is arbitrary, and may be any polygonal column shape, round column shape, or elliptical column shape. The side walls of the projections in FIGS. 4 through 6 and FIG. 7A are substantially perpendicular to the surface of the infrared absorption film, but the shape of the side walls of the projections may by modified by adjusting the dry etching conditions. For example, it is possible to use a pyramidal or conical shape as shown in FIG. 7B, a shape in which the distal end portion is removed from a pyramid or cone as shown in FIG. 7C, a hemispherical shape in which the angle is rounded as shown in FIG. 7D, or another shape. In a structure in which the side walls are tilted in this manner, reflection off the top portion of the projection can be minimized, thereby enabling further increased infrared absorption efficiency.

The method for manufacturing the thermal-type infrared detection element structured as shown in FIG. 4 will next be described with reference to FIGS. 8 through 15. The structures or manufacturing methods described below are merely examples, and the manufacturing conditions, film thickness, and other conditions may be modified as appropriate.

First, as shown in FIG. 8, a CMOS circuit (reading circuit 2) or the like for reading a signal is formed inside a silicon wafer or other circuit board 1 using a publicly known method. A metal such as Al, Ti, or W, or a silicide of any of these metals is then deposited as a film or the like with a thickness of about 500 nm on the circuit board 1 by RF sputtering and is partially etched using as a mask a resist pattern formed using a photolithography technique, and an infrared reflection film 3 for reflecting incident infrared rays to the photoreceptor 11 of each pixel is formed. A contact unit 14 is also formed for connecting the reading circuit 2 in the circuit board 1 with one end of the wiring whose other end is connected to the bolometer layer. A silicon oxide film, silicon nitride film, silicon oxynitride film, or the like is then deposited using a plasma CVD method, and a protective film 4 for protecting the infrared reflection film 3 and contact unit 14 is formed.

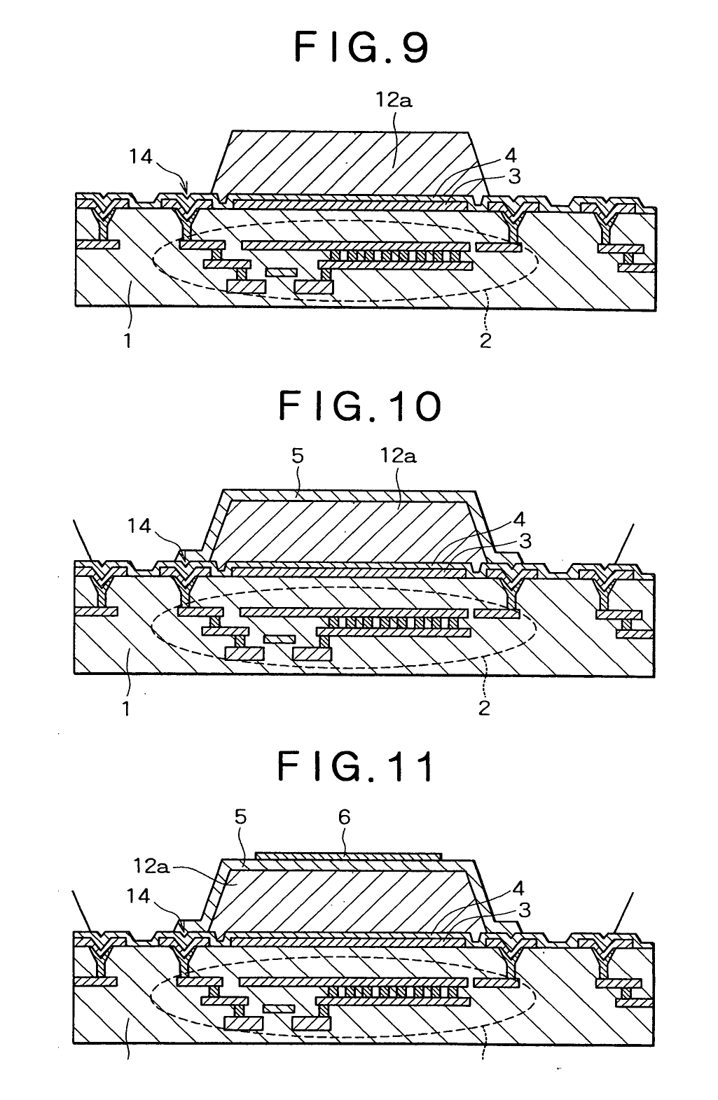

As shown in FIG. 9, a photosensitive polyimide film or other organic film is then applied over the entire surface of the circuit board 1, and the photosensitive polyimide film is removed by exposure/development from the area other than the area in which the photoreceptor 11 is formed. Densification is then performed at a temperature of about 400° C., and a sacrifice layer 12a for forming a microbridge structure is formed. The thickness of this sacrifice layer 12a is set to about 1.2 μm so that an optical resonance structure is formed by the infrared reflection film 3 and the photoreceptor 11 described hereinafter.

As shown in FIG. 10, a film having a thickness of about 300 nm is then formed over the entire surface of the circuit board 1 from a material selected from the group consisting of SiCO, SiO, SiN, SiC, SiON, SiCN, and the like using RF sputtering or plasma CVD, and a first infrared absorption film 5 is formed on the top portion and side surface of the sacrifice layer 12a. Any material from among the materials described above may be used to form the first infrared absorption film 5, but a material is preferably selected that is resistant to the etching of the sacrifice layer 12a described hereinafter, has adequate strength to support the bolometer layer 6 formed thereon, and has good adhesion to and no interaction with the bolometer layer 6. An SiCO, SiC, SiCN, or other carbon-containing film can be formed by either an RF sputtering method or a plasma CVD method, and an SiO, SiN, SiON, or other carbon-free film can be formed by a plasma CVD method.

As shown in FIG. 11, Vanadium oxide is then deposited on the first infrared absorption film 5 by reactive sputtering in an oxygen atmosphere, the vanadium oxide thin film is partially etched by plasma etching using fluorine gas with a resist pattern as the mask, and a bolometer layer 6 is formed on the first infrared absorption film 5. A vanadium oxide thin film is used as the bolometer layer 6 herein, but another material having a large temperature coefficient of resistance (TCR) as described above may also be used.

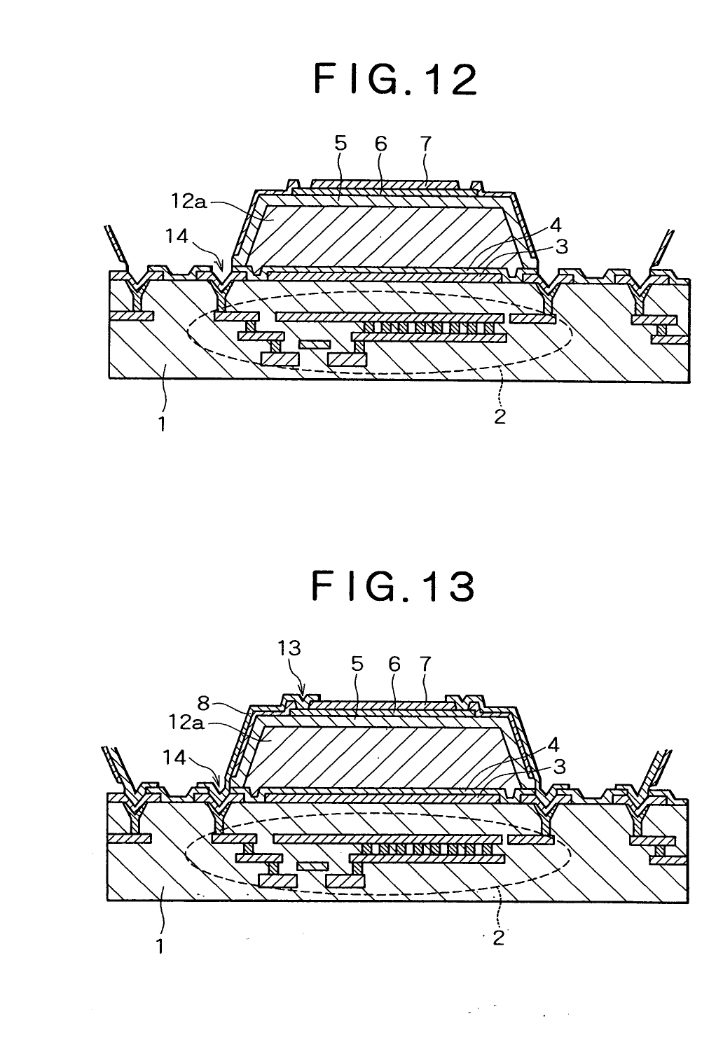

As shown in FIG. 12, a film having a thickness of about 50 nm is then formed over the entire surface of the circuit board 1 from a material selected from the group consisting of SiCO, SiO, SiN, SiC, SiON, SiCN, and the like using RF sputtering or plasma CVD, and a second infrared absorption film 7 for protecting the bolometer layer 6 is formed. Any material from among the materials described above may be used to form the second infrared absorption film 7, but a material is preferably selected that is different from that of the first infrared absorption film 5 in order to improve the infrared absorption characteristics, and that has good adhesion to and no interaction with the bolometer layer 6. Plasma etching is then performed using carbon tetrafluoride as the etching gas and a resist pattern as the mask, the first infrared absorption film 5 and second infrared absorption film 7 on the contact unit 14 are removed, the second infrared absorption film 7 on the end portion of the bolometer layer 6 is removed, and an electrode unit 13 is formed.

As shown in FIG. 13, a film is then formed from Ti, Ti alloy, NiCr, or another wiring metal by RF sputtering, after which the wiring metal is partially etched by plasma etching using a gas mixture of chlorine and boron trichloride with a resist pattern as the mask, and wiring 8 is formed. This wiring 8 electrically connects the electrode unit 13 of the bolometer layer 6 with the contact unit 14 of the circuit board 1, and serves as the beam 10 for holding the photoreceptor 11 in midair.

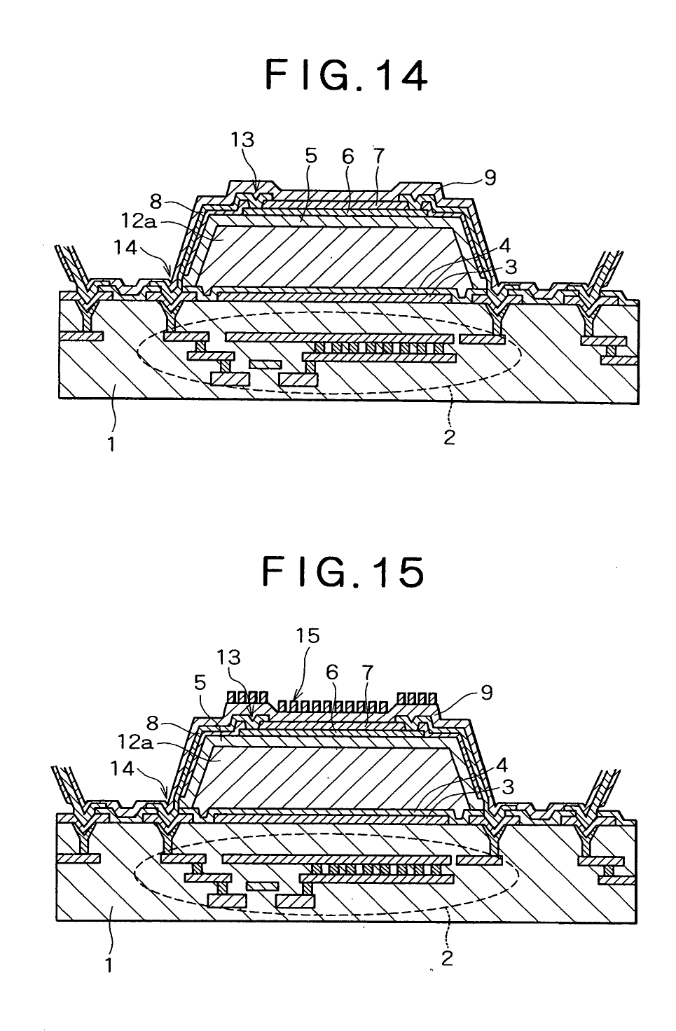

As shown in FIG. 14, a film having a thickness of about 300 nm is then formed over the entire surface of the circuit board 1 from a material selected from the group consisting of SiCO, SiO, SiN, SiC, SiON, SiCN, and the like by RF sputtering or plasma CVD, and a third infrared absorption film 9 for protecting the bolometer layer 6 and the wiring 8 is formed. Any material from among the materials described above may be used to form the third infrared absorption film 9, but a material is preferably selected that is different from that of the first infrared absorption film 5 and second infrared absorption film 7 in order to improve the infrared absorption characteristics, that has good environmental resistance in order to withstand being exposed on the outermost surface, and that will act as an etching stopper for the convex pattern 15 described hereinafter.

As shown in FIG. 15, a film having a thickness of about 500 nm is then formed over the entire surface of the circuit board 1 from a material selected from the group consisting of SiCO, SiO, SiN, SiC, SiON, SiCN, and the like by RF sputtering or plasma CVD, after which a resist pattern is formed thereon in which a pattern of squares having an edge length of approximately 0.5 μm is arranged at a pitch of approximately 0.5 μm using, for example, an i-line stepper; plasma etching is performed using carbon tetrafluoride as the etching gas with this resist pattern as the mask; and a convex pattern 15 is formed in which projections having an aspect ratio of 1 or higher are arranged at a pitch of approximately 0.5 μm. Any of the abovementioned materials may be used to form this convex pattern 15, but a material is preferably selected that high etching selectivity with respect to the third infrared absorption film 9, and that has good environmental resistance in order to withstand being exposed on the outermost surface.

Plasma etching is then performed using carbon tetrafluoride as the etching gas and a resist pattern as the mask; a through hole (not shown in the drawing) is formed through the first infrared absorption film 5, second infrared absorption film 7, and third infrared absorption film 9; and the sacrifice layer 12a is removed using an ashing device. As shown in FIG. 4, a thermal-type infrared detection element is formed having a microbridge structure in which the photoreceptor 11 is brought into contact with the circuit board 1 only by the beam 10.

By the thermal-type infrared detection element of the present embodiment thus configured, since a convex pattern 15 in which a plurality of substantially congruent projections composed of an infrared absorbing material are arranged at a substantially constant pitch is formed on the side of the photoreceptor 11 that is irradiated with infrared rays, reflection of infrared rays on the flat surface of the third infrared absorption film 9 is minimized, and the absorption efficiency of infrared rays can be increased. The sensitivity of the thermal-type infrared detection element can thereby be enhanced. Since this convex pattern 15 can also be formed using a common semiconductor manufacturing device, and is endowed with excellent adhesion to the immediately underlying infrared absorption film (the third infrared absorption film 9 in this case), the reliability and uniformity of the thermal-type infrared detection element can be enhanced.

The convex pattern 15 is formed on the third infrared absorption film 9 in the embodiment described above, but it is sufficient if the convex pattern 15 is formed on the side of the photoreceptor 11 that is irradiated with infrared rays in the present invention. For example, the third infrared absorption film 9 may be omitted, and the convex pattern 15 may be formed on the second infrared absorption film 7 as shown in FIG. 16, or the convex pattern 15 may be formed in the second infrared absorption film 7 itself. Another film besides the first infrared absorption film 5, second infrared absorption film 7, and third infrared absorption film 9 may also be added.

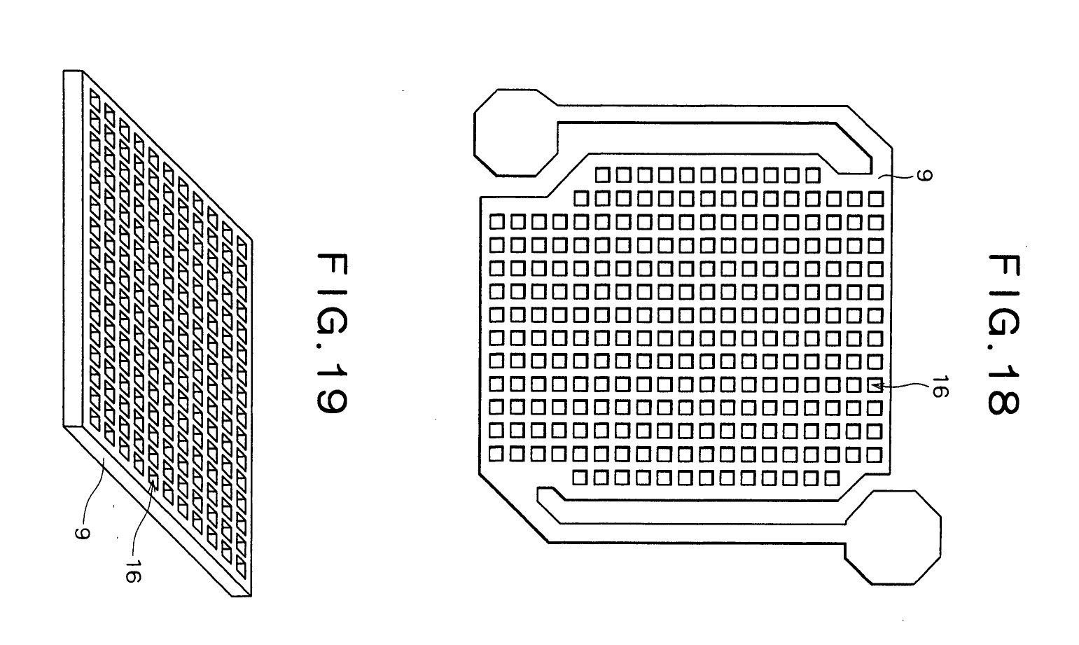

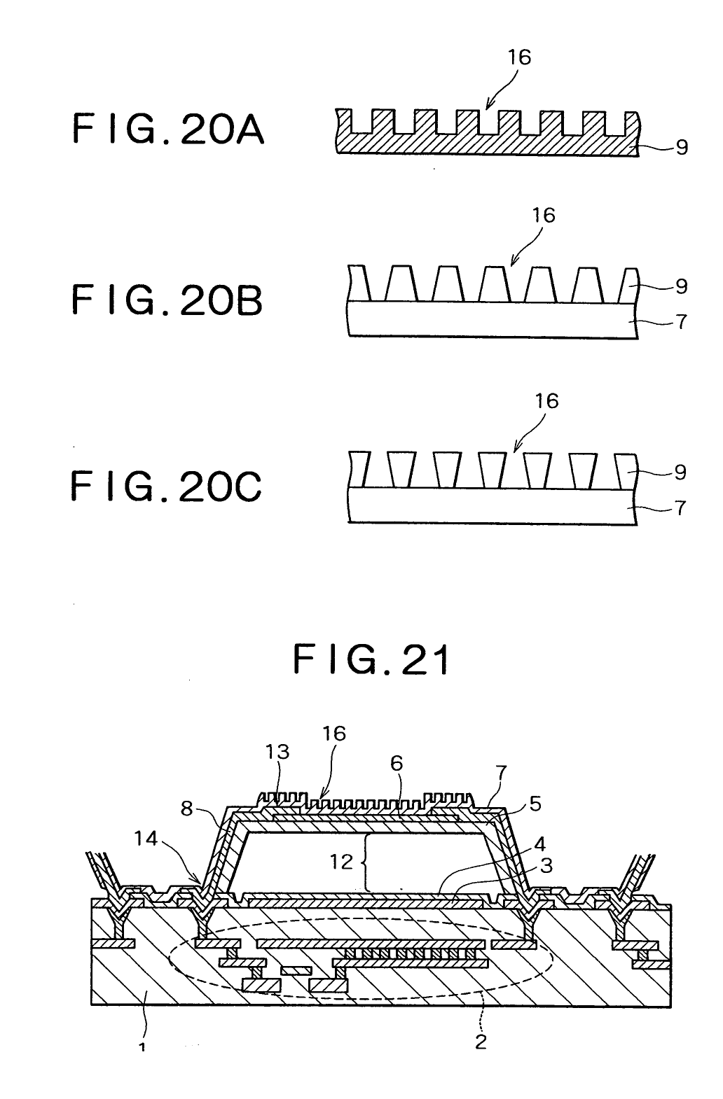

The thermal-type infrared detection element according to a second embodiment of the present invention will next be described with reference to FIGS. 17 through 21. FIG. 17 is a sectional view in which one pixel of the thermal-type infrared detection element of the present embodiment is depicted in alignment with the current path; FIG. 18 is a schematic plane view of the structure of the concave pattern; and FIG. 19 is a perspective view thereof. FIG. 20 is a sectional view showing variation in the shape of the concave pattern; and FIG. 21 is a sectional view showing another structure of the thermal-type infrared detection element of the present embodiment.

The convex pattern 15 was formed on the side of the photoreceptor 11 that is irradiated with infrared rays in the first embodiment described above, but incident infrared rays can be scattered and prevented from reflecting if the surface is not flat, and since the heat capacity of the photoreceptor 11 increases by an amount commensurate with the area of the convex pattern 15 when the convex pattern 15 is formed, a concave pattern 16 is formed instead of the convex pattern 15 in the present embodiment.

More specifically, in the thermal-type infrared detection element of the present embodiment, a photoreceptor 11 composed of a bolometer layer 6 formed from vanadium oxide or another material and an infrared absorption body is held in midair by a beam 10 composed of wiring 8 formed from Ti or the like and a protective film for protecting the wiring 8, and a concave pattern 16 for dispersing the incident infrared rays and increasing the absorption efficiency is formed in an infrared absorption film (the third infrared absorption film 9 in this case) disposed on the side of the photoreceptor 11 that is irradiated with infrared rays, as shown in FIG. 17.

The concave pattern 16 is shown schematically in FIGS. 18 and 19, and is composed of a plurality of substantially congruent (rectangular in horizontal cross-section in this case) holes arranged in a matrix at a substantially constant pitch. This concave pattern 16 may be formed by a variety of methods, and can easily be formed by a process in which a resist pattern (a pattern of hollow rectangles in this case) having a prescribed shape is formed on the infrared absorption film (third infrared absorption film 9) of the outermost layer, and the third infrared absorption film 9 is dry-etched using the resist pattern as a mask.

As in the first embodiment, any appropriate shape or pitch may be set for the holes, but it is preferred in order to increase the infrared absorption efficiency that the aspect ratio (depth/width ratio of the holes) be increased and that the interval between adjacent holes be reduced within ranges that allow formation by common photolithography techniques and dry etching techniques. It has been confirmed according to the knowledge of the present applicant that the infrared absorption efficiency is dramatically improved by making the aspect ratio equal to 1 or higher, and making the interval between adjacent holes smaller than the depth of the holes.

The concave pattern 16 may be formed so as to penetrate through the infrared absorption film (the third infrared absorption film 9 in this case) of the outermost layer, or may be formed by etching partway into the infrared absorption film of the outermost layer as shown in FIG. 20A.

The cross-section in the horizontal direction of the holes that constitute the concave pattern 16 is square in FIGS. 18 and 19, but the shape of the holes is arbitrary and the cross-section thereof may be any polygonal shape, round shape, or elliptical shape. The side walls of the holes in FIGS. 17 through 19 and FIG. 20A are substantially perpendicular to the surface of the infrared absorption film, but the shape of the side walls of the holes may by modified by adjusting the dry etching conditions. For example, the side walls may be tilted so as to create a forward-tapered shape in which the width at the top is greater than the width at the bottom, as shown in FIG. 20B, or tilted so as to create an inverse tapered shape in which the width at the top is smaller than the width at the bottom, as shown in FIG. 20C. A shape may also be created in which the angles are rounded.

The method for manufacturing the thermal-type infrared detection element having the structure described above will next be described. First, the infrared reflection film 3 and protective film 4 are formed using the same method used in the first embodiment on the circuit board 1 in which the reading circuit 2 is formed, and the sacrifice layer 12a composed of a photosensitive polyimide film or the like is formed in the area in which the photoreceptor 11 is formed. Next, after the first infrared absorption film 5 is formed on the top portion and side surface of the sacrifice layer 12a, vanadium oxide is deposited on the top portion of the sacrifice layer 12a and the bolometer layer 6 is formed, after which the second infrared absorption film 7 for protecting the bolometer layer 6 is formed. A film of Ti, Ti alloy, NiCr, or another wiring metal is then formed, the electrode unit 13 of the bolometer layer 6 is electrically connected with the contact unit 14 of the circuit board 1, and the wiring 8 for holding the photoreceptor 11 in midair is formed (see FIGS. 8 through 13).

A film having a thickness of about 500 nm is then formed over the entire surface of the circuit board 1 from a material selected from the group consisting of SiCO, SiO, SiN, SiC, SiON, SiCN, and the like by RF sputtering or plasma CVD, and the third infrared absorption film 9 for protecting the bolometer layer 6 and wiring 8 is formed. Any material from among the materials described above may be used to form the third infrared absorption film 9, but a material is preferably selected that high etching selectivity with respect to the second infrared absorption film 7, and that has good environmental resistance in order to withstand being exposed on the outermost surface. A resist pattern is then formed thereon in which a pattern of open squares having an edge length of approximately 0.5 μm is arranged at a pitch of approximately 0.5 μm using, for example, an i-line stepper; plasma etching is performed using carbon tetrafluoride as the etching gas with this resist pattern as the mask; and a concave pattern 16 is formed in which holes having an aspect ratio of 1 or higher are arranged at a pitch of approximately 0.5 μm.

A through hole (not shown in the drawing) is then formed through the first infrared absorption film 5, second infrared absorption film 7, and third infrared absorption film 9; and the sacrifice layer 12a is removed using an ashing device. As shown in FIG. 17, a thermal-type infrared detection element is formed having a microbridge structure in which the photoreceptor 11 is brought into contact with the circuit board 1 only by the beam 10.

By the thermal-type infrared detection element of the present embodiment thus configured, a concave pattern 16 in which a plurality of substantially congruent holes are arranged at a substantially constant pitch is formed in an infrared absorption film disposed on the side of the photoreceptor 11 that is irradiated with infrared rays. Therefore, reflection of infrared rays in the third infrared absorption film 9 is minimized, and the absorption efficiency of infrared rays can be increased. The sensitivity of the thermal-type infrared detection element can thereby be enhanced. Since this concave pattern 16 can also be formed using a common semiconductor manufacturing device, and has excellent adhesion to the immediately underlying infrared absorption film (the second infrared absorption film 7 in this case), the reliability and uniformity of the thermal-type infrared detection element can be enhanced. The heat capacity of the photoreceptor 11 can also be reduced in comparison to the structure of the first embodiment in which the convex pattern 15 is formed. The sensitivity of the thermal-type infrared detection element can therefore be further enhanced.

The concave pattern 16 is formed in the third infrared absorption film 9 in the embodiment described above, but it is sufficient in the present invention if the concave pattern 16 is formed in an infrared absorption film disposed on the side of the photoreceptor 11 that is irradiated with infrared rays. For example, the third infrared absorption film 9 may be omitted, and the concave pattern 16 may be formed in the second infrared absorption film 7, as shown in FIG. 21. Another film besides the first infrared absorption film 5, second infrared absorption film 7, and third infrared absorption film 9 may also be added.

A convex pattern 15 is formed in the infrared absorption body in the first embodiment, and a concave pattern 16 is formed in the infrared absorption body in the second embodiment, but these configurations may also be combined. For example, projections may also be formed in the portions not occupied by holes in an infrared absorption film in which the concave pattern 16 is formed.

A thermal-type infrared detection element having a structure in which the photoreceptor 11 is held in midair by the beam 10 was described in the embodiments above, but the present invention is in no way limited by these embodiments, and can be applied in the same manner to a thermal-type infrared detection element in which a thermal separation structure is formed by hollowing out the substrate underneath the photoreceptor.

The structure of the present invention is not limited to a infrared absorption body that constitutes the photoreceptor of a thermal-type infrared detection element, and may also be applied to a general structure for efficiently absorbing infrared rays. For example, the present invention may be utilized as an anti-reflective material formed on the surface of a solar cell, or as a band-pass filter or other filter material.

What is claimed is:

1. A thermal-type infrared detection element comprising:
a substrate;
a photoreceptor provided with a heat-sensitive resistor and an infrared absorption body;
a beam having one end fixed to said substrate, for holding said photoreceptor in midair; and
a convex pattern formed on the side of said photoreceptor that is irradiated with infrared rays, in which a plurality of substantially congruent projections composed of an infrared absorbing material are arranged at a substantially constant pitch.

2. A thermal-type infrared detection element comprising:
a substrate;
a photoreceptor having a heat-sensitive resistor and an infrared absorption body; and
a beam having wiring whose one end is connected to said heat-sensitive resistor and whose other end is connected to a circuit formed in a substrate, for holding said photoreceptor in midair; wherein
said infrared absorption body comprises:
a first infrared absorption film formed in the bottom layer of said heat-sensitive resistor;
a second infrared absorption film formed in the top layer of said heat-sensitive resistor;
a third infrared absorption film formed in the top layer of said wiring connected to said heat-sensitive resistor via a through-hole provided to said second infrared absorption film; and
a convex pattern formed in the top layer of said third infrared absorption film, wherein a plurality of substantially congruent projections composed of an infrared absorbing material are arranged at a substantially constant pitch.

3. The thermal-type infrared detection element according to claim 1, wherein the height-to-width ratio of said projections is 1 or higher, and the interval between adjacent said projections is smaller than said height.

4. The thermal-type infrared detection element according to claim 2, wherein the height-to-width ratio of said projections is 1 or higher, and the interval between adjacent said projections is smaller than said height.

5. A thermal-type infrared detection element comprising:
a substrate;
a photoreceptor having a heat-sensitive resistor and an infrared absorption body;
a beam for holding the photoreceptor in midair, whose one end is fixed to the substrate; and
a concave pattern formed in an infrared absorption film disposed on the side of said photoreceptor that is irradiated with infrared rays, in which a plurality of substantially congruent holes are arranged at a substantially constant pitch.

6. A thermal-type infrared detection element comprising:
a substrate;
a photoreceptor having a heat-sensitive resistor and an infrared absorption body; and
a beam having wiring whose one end is connected to said heat-sensitive resistor and whose other end is connected to a circuit formed in a substrate, for holding said photoreceptor in midair; wherein
said infrared absorption body comprises:
a first infrared absorption film formed in the bottom layer of said heat-sensitive resistor;
a second infrared absorption film formed in the top layer of said heat-sensitive resistor; and
a third infrared absorption film that is formed in the top layer of said wiring connected to said heat-sensitive resistor via a through-hole provided to said second infrared absorption film, and that is provided with a concave pattern in which a plurality of substantially congruent holes are arranged at a substantially constant pitch.

7. The thermal-type infrared detection element according to claim 5, wherein the depth-to-width ratio of said holes is 1 or higher, and the interval between adjacent holes is smaller than said depth.

8. The thermal-type infrared detection element according to claim 6, wherein the depth-to-width ratio of said holes is 1 or higher, and the interval between adjacent holes is smaller than said depth.
